(19) United States
(12) Patent Application Publication
(10) Pub. No.: US 2018/0055476 A1
(43) Pub. Date: Mar. 1, 2018

(54) SYSTEM AND METHOD FOR PRE-SURGICAL BREAST TUMOR LOCALIZATION USING NON-RADIOACTIVE LOCALIZATION MATERIAL

(71) Applicant: MAYO Foundation for Medical Education and Research, Rochester, NY (US)

(72) Inventors: Victor J. Pizzitola, Scottsdale, AZ (US); William Pavlicek, Scottsdale, AZ (US)

(21) Appl. No.: 15/694,284

(22) Filed: Sep. 1, 2017

Related U.S. Application Data
(60) Provisional application No. 62/382,694, filed on Sep. 1, 2016.

(51) Int. Cl. A61B 8/08; A61B 8/00; A61B 90/00

(52) U.S. Cl. CPC A61B 8/0833; A61B 2090/3925; A61B 90/39; A61B 8/461

(57) ABSTRACT

A system and method is provided that includes a method for controlling an ultrasound detector system for localizing a marker seed at least partially composed of a piezoelectric material. The method includes arranging least one marker seed including a piezoelectric material proximate to a region of interest (ROI) in a subject. The method also includes arranging a detector probe including an ultrasound transducer to direct ultrasound pulses into the subject, detect return signals generated by the marker seed in response to receiving the ultrasound pulses, and generate localization data based on the return signals detected. The method further includes utilizing a display communicating a location of the marker seed based on the localization data to locate the marker seed and perform a medical procedure on the ROI in the subject.

### CROSS-REFERENCE TO RELATED APPLICATIONS

This application is based on, claims the benefit of, and incorporates herein by reference in its entirety, U.S. Provisional Patent Application Ser. No. 62/382,694 filed Sep. 1, 2016, and entitled “Pre-surgical Breast Tumor Localization Using Non-Radioactive Localization Material.”

### STATEMENT REGARDING FEDERALLY SPONSORED RESEARCH

N/A

### BACKGROUND

Breast tumor localization is a radiologic procedure that is utilized prior to surgical excision of breast cancer. Breast surgeons rely on this procedure for the accurate localization and subsequent excision at surgery. Before localization techniques were created, surgical excision of tumors was by manual palpation or total mastectomy.

The most common tumor localization technique in use today is wire localization, whereby radiologists use mammogram or ultrasound guidance to place metallic wires next to the legion requiring surgical excision. The surgeon then uses the wire as a guide during surgery. This technique has limitations because it must be placed on the same day of surgery. Accordingly, any difficulties encountered by the radiologist in localizing the lesion leads to delays in surgery as the surgeon waits for the radiologist to complete the localization. Additionally, the patient leaves the radiology department with either a wire or a wire and needle and must travel to the operating room with the device sticking out of her breast. In some situations, the wire could move or be displaced during travel, which can result in additional surgery delays.

An alternative technique was developed that uses radioactive I-125 seeds to localize breast tumors for surgical excision. The seed can be placed days prior to surgery, eliminating the need for the operating room to wait for the radiologist to complete the procedure. The seed is placed into the breast and no wire is, therefore, extending outside the breast. The surgeon then uses a Geiger counter to find the seed during surgery and guide excision.

The advantages of seed localization have been accepted by the radiology and surgery communities. However, the radioactive nature of the seed presents many logistical and regulatory barriers to implementation and adoption. Most outpatient clinics are not equipped to handle the nuclear regulations required by radioactive seed programs. The current use of radioactive iodine seed localizations by their nature are tightly controlled material and can result in radiation exposure to the physician and others involved in handling the radioactive seeds. The radioactive nature of the seed requires many careful steps in the receipt, placement, removal, and processing of the seed. Radioactive seeds require the radiology staff, surgical staff, and pathology staff to be well-trained in the handling and disposal of radioactive materials. This can be costly and prevents adoption of seeds.

Thus, regulation can be costly and tedious, such that many clinics and departments do not want to deal with the associated regulations and controls. Also, nuclear techniques may be feared by medical personnel and even patients. In the case of patients, the fear may prevent them from even considering the technique.

Accordingly, what is needed is an improved system and method for breast tumor localization that is efficient and limits radiation exposure.

### SUMMARY OF THE DISCLOSURE

The present disclosure addresses the aforementioned drawbacks by providing systems and methods for marking the location and extent of an anatomical region-of-interest, such as a tumor, using non-radioactive marker seeds. For example, the seeds may be compose at least partially, of piezoelectric material. The position and orientation of the marker seeds can be measured or otherwise detected using a detection device. For example, the detection device may include an ultrasound transducer.

In accordance with one aspect of the present disclosure, an ultrasound detector system is provided for localizing a marker seed at least partially composed of a piezoelectric material. The system includes a detector probe comprising a housing extending along a central axis from a distal end to a proximal end and an ultrasound transducer arranged at the distal end of the housing. The transducer is configured to generate ultrasound pulses into a subject having received the marker seed in a region of interest (ROI), detect return signals generated by the marker seed in response to receiving the ultrasound pulses, and generate localization data based on the return signals detected. The system includes a processor in communication with the ultrasound transducer to receive the localization data therefrom and configured to process the localization data to compute a location of the marker seed. The system also includes an output communicating the location of the marker to a user based on the computed location of the marker seed.

In accordance with another aspect of the present disclosure, a method is disclosed for controlling an ultrasound detector system for localizing a marker seed at least partially composed of a piezoelectric material. The method includes arranging least one marker seed including a piezoelectric material proximate to a region of interest (ROI) in a subject and arranging a detector probe including an ultrasound transducer to direct ultrasound pulses into the subject, detect return signals generated by the marker seed in response to receiving the ultrasound pulses, and generate localization data based on the return signals detected. The method includes utilize a display communicating a location of the marker seed based on the localization data to locate the marker seed and perform a medical procedure on the ROI in the subject.

In accordance with yet another aspect of the present disclosure, a kit is disclosed for localizing a marker seed at least partially composed of a piezoelectric material. The kit includes at least one marker seed including a piezoelectric material and a detector probe. The probe includes a housing extending along a central axis from a distal end to a proximal end, an ultrasound transducer arranged at the distal end of the housing. The ultrasound transducer configured generate ultrasound pulses into a subject having received the marker seed in a region of interest (ROI), detect return signals generated by the marker seed in response to receiving the ultrasound pulses, and generate localization data based on the return signals detected. The kit also includes a processor in communication with the ultrasound transducer to receive the localization data therefrom and configured to process the localization data to compute a location of the marker seed and an output communicating the location of the marker to a user based on the computed location of the marker seed.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a schematic diagram of a system for use in a medical procedure in accordance with the present disclosure.

FIG. 2 is a schematic illustration of a marker seed and tool in accordance with the present disclosure.

FIG. 3 is an illustration of a computer system of the system of FIG. 1.

FIG. 4 is a flow chart in accordance with one aspect of the disclosure.

FIG. 5 is a flow chart in accordance with another aspect of the disclosure.

### DETAILED DESCRIPTION

According to the systems and methods described in the present disclosure, one or more non-radioactive marker seeds may be implanted to mark and define the center and extent of an anatomical region-of-interest, such as a tumor or other lesion. Using an ultrasound-based localization system, the location of the marker seeds may be accurately identified. When marking the location of a breast tumor, the clinician may plan out a surgery, using the marker seed(s), to allow for the best achievable cosmetic result, while ensuring optimal oncologic outcomes. The marker seeds may be implanted into a subject to mark the center, boundaries, or both, of an anatomical region-of-interest, such as a tumor. In one non-limiting example, the marker seeds may be implanted to mark the boundary of a breast tumor; however, other clinical applications will be apparent to those skilled in the art.

In an effort to overcome the barriers to adoption of radioactive seed localization, a non-radioactive, biologically-favorable material may be used that may allow for safe placement into the breast for pre-surgical excision. A hand-held device may be used by the surgeon to localize the tumor. Current radioactive seed removal techniques and Geiger probes do not provide the surgeon accurate details of the distance of the lesion from the hand-held device. The hand-held device may be configured to display distance data to the surgeon. This data may aid the surgeon in locating the tumor.

Separately, a localizing tool may be included within the system. The localization tool may be used to interrogate and locate the micro-implant (marker seed) using, for example, ultrasound. The tool may incorporate the requisite properties suitable for sterile surgical intervention. The hand-held tool may provide range (distance) to the micro-implant, a system of targetry (direction) and/or a method of audible tone to the surgeon whose audible intensity may be altered depending upon the correctness of the direction and distance to the micro-implant.

A method of quickly targeting the lesion may provide an enhanced understanding of the exact position of the tissue to be excised in relation to the surgeon's approach. This system of targeting is intended as an aid for the surgeon for the removal of the tissue.

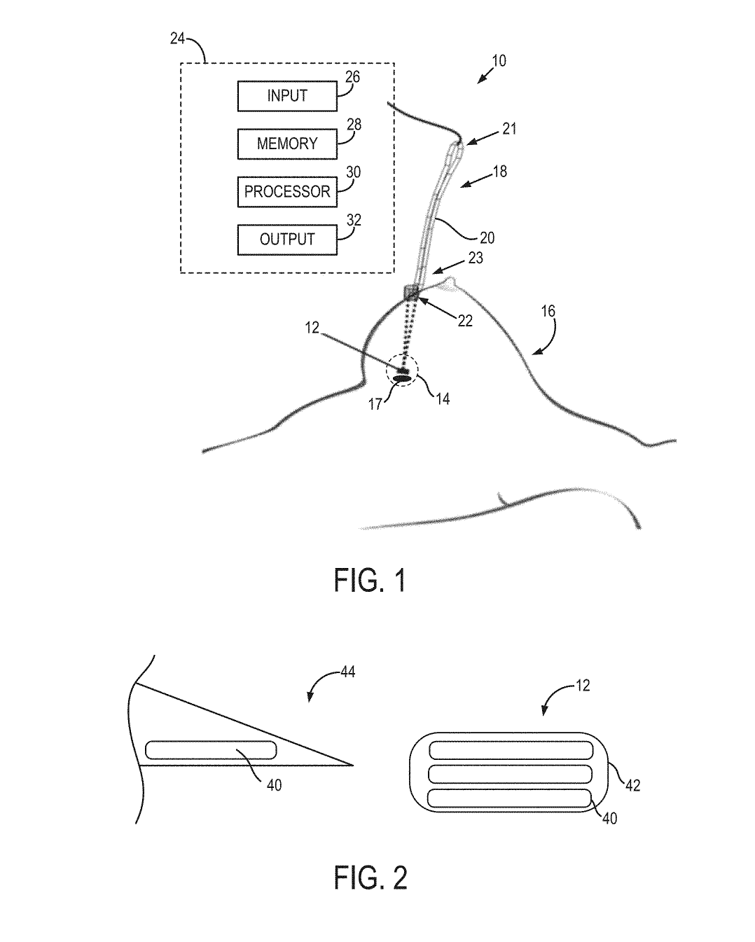

As shown in FIG. 1, an example marker seed localization system 10 is shown. The system 10 includes one or more marker seeds 12 that are implanted into an anatomical region-of-interest 14 in a subject 16. The region-of-interest 14 may include a tumor 17. In some applications, one or more of the marker seeds 12 may also be positioned on a skin surface of the subject 16.

A detector 18 may be used to detect or otherwise measure the position, orientation, or both, of the marker seeds 12. The detector 18 may include a housing 20 that contains an ultrasound transducer 22. The housing 20 may define a hand-held structure, such that the detector 18 can be held and used by a clinician in an operating room or other surgical or clinical environment. As one example, the housing 20 can extend from a proximal end 21 to a distal end along an axis 23. The ultrasound transducer 22 may be positioned or otherwise arranged at the distal end 23 of the housing 20.

The detector 18 may be in electrical communication with a computer system 24, which generally operates the detector 18 and receives signal data from the ultrasound transducer 22, such as via an input 26. The computer system 24 includes the input 26, a memory 28, at least one hardware processor 30, and an output 32. The computer system 24 may provide auditory feedback, visual feedback, or both, to a surgeon to assist the surgeon during a procedure. This feedback may be provided via the output 32, which may include a speaker, a display, or the like. Thus, the computer system 24 can be generally implemented with a hardware processor 30 and a memory 28. The one or more processors 30 receive signal data from the ultrasound transducer 22 via the input 26, and processes the signal data to detect or otherwise measure a position, orientation, or both, of the marker seeds 12. In some configurations, the computer system 24 can be arranged within the housing 20 of the detector 18; however, in other configurations the computer system 24 is physically separate from the detector 18.

An example marker seed 12 that can be implemented in accordance with the present disclosure is illustrated in FIG. 2. In the example shown in FIG. 2, the marker seed 12 has a generally cylindrical shape; however, it will be appreciated that any other suitable shapes can be implemented, including spherical shapes, ellipsoidal shapes, rectangular shapes, and so on. Each marker seed 12 can be sized to fit in standard needles for implantation.

In general, the marker seeds 12 are composed at least partially of a piezoelectric material 40 that may be encapsulated in an optional bio-compatible material 42. For instance, the marker seeds 12 can be constructed to have physical properties that incorporate piezoelectric behavior in the ultrasound range of frequencies, such that the piezoelectric material 40 is excited by ultrasound energy delivered form the ultrasound transducer 22 of the system 10 of FIG. 1 to produce signals that are received by the ultrasound transducer 22 and processed by the processor 30 of the computer 24 to localize the marker seed 12. The marker seeds 12 are capable of being placed by a physician in the desired location of the breast or other organ system using a needle. Similarly, a surgical tool 44 may optionally be formed to include piezoelectric material 40. In this way, as will be described, a user may utilize the system 10 to image not only the marker seed(s) 12, but also the surgical tools 44.

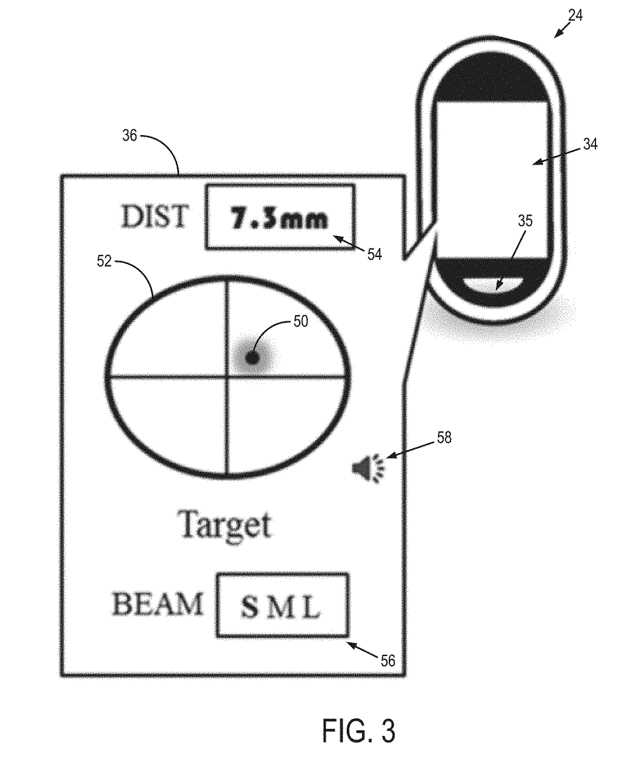

As mentioned above, in some configurations the computer system 24 used to control the detector 18 and to process signal data to localize the marker seeds 12 is physically separate from the housing 20 of the detector 18. For example, referring to FIG. 3, the computer system 24 can be a small hand-held device, and may include a phone, tablet or other computer system. The computer system 24 can include an integrated display 34 to provide visual feedback to the user. The computer system 24 can also include a speaker 3 for providing auditory feedback to the user.

The integrated display 34 can display reports, such as via a graphical user interface (“GUI”) 36, which may also be used to control the detector 18 and to display data and other feedback to the user. In some implementations, the integrated display 34 can include a touch-screen, such that the user can input commands via the integrated display 34. In this manner, the integrated display 34 can provide both the input 28 and the output 32 for the computer system 24. As one example, the computer system 24 can be a smart phone, tablet, or other such hand-held computing device.

As illustrated, the GUI 36 may give a graphical illustration or map showing the target or seed 50 within a region-of-interest (ROI) or region-of-display 52. In additional to illustrations, the GUI 36 may include text indicators, such as a measured distance 54 to the target 50 and control or delivery parameters of the ultrasound beam 56. Furthermore, the GUI 36 may include additional operational controls, including an audio control 58. Though illustrated in this non-limiting example, as integrated into the display 34 of the computer system 24, as mentioned above, the GUI 36 generated by the computer system 24 can display distance data to the user. This data may aid the surgeon in locating the anatomical ROI 14, such as a tumor or lesion.

In operation, the above-described system 10 generally operates by interrogating the volume around the tip of the detector 18 for a marker seed 12. The echoes generated by exciting the piezoelectric materials 40 in the marker seed 12 with the ultrasound transducer 22 in the detector 18 may then be measured and used to determine the distance of the marker seed 12 from the tip of the detector 18. The distance calculation provides a method of feedback that is correlated to both a visual display 34 and, if desired, auditory feedback. As described above, directionality can also be measured and displayed via a map 52.

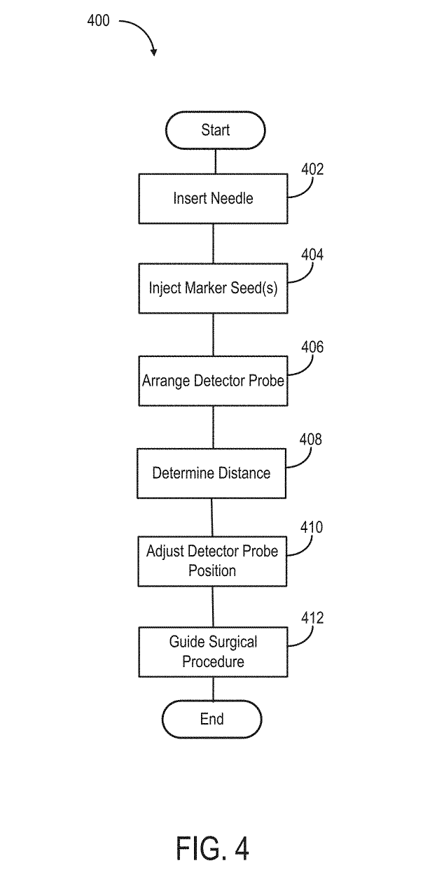

More particularly, referring now to FIG. 4, a flowchart is illustrated that provides some steps of a non-limiting example method 400 for using the above-described detector system. In some configurations, the ROI may correspond to or include a tumor or legion. At process block 402, the method 400 may include inserting a needle into a patient. The insertion point of the needle may be selected relative to the ROI such that, at process block 404, the needle can be used to inject at least one marker seed into the patient. In some applications, it may be desirable to inject a plurality of marker seeds. The injection site may be in or near the ROI or may include or surround the ROI.

Still referring to FIG. 4, the method may include arranging a detector probe relative to the patient, according to process block 406. The insertion site, similarly to this site, may be near the ROI. In some situations, it may be beneficial to be substantially distant from the ROI due to anatomical features or other limitations. At process block 408, the detector probe may detect the at least one marker seed, such as by exiting the injected marker seed using ultrasound selected to energize the piezoelectric material in the marker seed. As described above, a processor may be configured to determine a distance between the detector probe and the at least one marker seed using the signals emitted by the injected marker seed or seeds. The distance may be displayed to a user, such as a surgeon, in real-time on a display, such as described above.

At process block 410, the detector probe position may be adjusted. The user may utilize the displayed distance to determine an adjustment strategy. To determine an accurate distance between the at least one marker seed and the detector, a localization system may excite elements of the at least one marker seed and analyze received feedback via distance algorithms. This calculation may be performed by the processor, and the distance may again be displayed to the user.

The surgeon may use the real-time feedback from the display to decrease the distance between the detector probe and the at least one marker seed. Using the displayed information, an interventional procedure, such as a surgical procedure performed on the ROI may be guided, as indicated at process block 412. For example, in a way similar to the use of traditional radioactive seeds to guide surgical processes, the above described marker seed(s) may be used with the system to guide a surgical procedure, such as a resection or the like. Also, as described above, though optional, some surgical tools may also be formed with piezoelectric materials that allow the surgical tools to be further imaged and displayed in a manner similar to the marker seed(s).

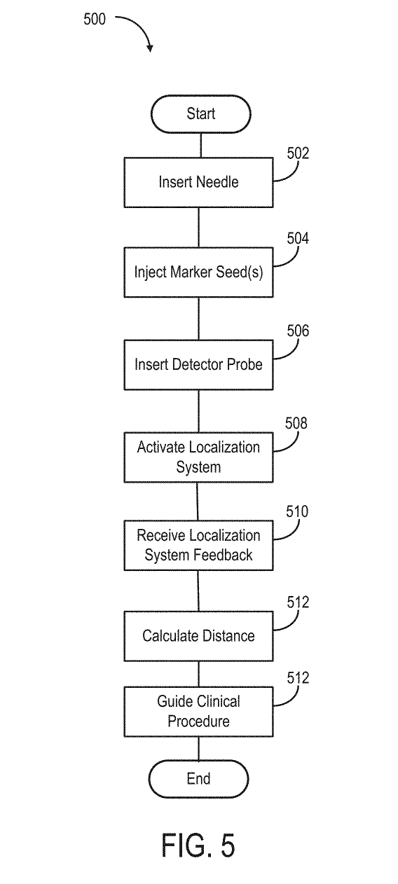

Referring to FIG. 5, a flowchart is illustrated as setting forth the steps of another example method 500 for using a detector to analyzing an ROI. At process block 502, a needle may be inserted into a patient. As described with respect to the method 400 of FIG. 4, the insertion site may be directed or selected relative to a region-of-interest. At process block 54, at least one marker seed may be injected into the patient. The needle may then be removed from the patient, and, immediately upon removal, a detector probe may be inserted into the patient, as indicated at process block 506. In some applications, a delay may occur between the needle removal and the detector probe. As described above, the insertion site may be near the ROI. Alternatively, the insertion site may be substantially distant from the region-of-interest.

Upon insertion of the detector probe, a localization system may be activated, according to process block 508. The localization system may be configured to transmit a signal through and/or near the ROI. The signal may reflect off of and/or energize a piezoelectric material of the at least one marker seed. Accordingly, at process block 510, the localization system may receive a feedback signal, whether reflected by or originating from the energized piezoelectric material of the marker seed(s). A processor may utilize the transmitted signal and the received signal to calculate a distance between the detector probe and the at least one marker seed, as indicated at block 512. The calculated distance may be displayed to a user, and may be used to adjust the position of the detector probe with respect to the region of interest (and at least one marker seed) and/or to guide a further clinical procedure, such as a surgical process, as indicated at process block 514.

The present disclosure has described one or more preferred embodiments, and it should be appreciated that many equivalents, alternatives, variations, and modifications, aside from those expressly stated, are possible and within the scope of the invention.

1. An ultrasound detector system for localizing a marker seed at least partially composed of a piezoelectric material, comprising:
a detector probe comprising:
a housing extending along a central axis from a distal end to a proximal end;
an ultrasound transducer arranged at the distal end of the housing and configured to:
generate ultrasound pulses into a subject having received the marker seed in a region of interest (ROI);
detect return signals generated by the marker seed in response to receiving the ultrasound pulses; and
generate localization data based on the return signals detected;
a processor in communication with the ultrasound transducer to receive the localization data therefrom and configured to process the localization data to compute a location of the marker seed; and
an output communicating the location of the marker to a user based on the computed location of the marker seed.

2. The system of claim 1 wherein the output includes a display configured to display a map of the ROI showing the computed location of the marker seed.

3. A method for controlling an ultrasound detector system for localizing a marker seed at least partially composed of a piezoelectric material, comprising:
arranging least one marker seed including a piezoelectric material proximate to a region of interest (ROI) in a subject;
arranging a detector probe including an ultrasound transducer to direct ultrasound pulses into the subject, detect return signals generated by the marker seed in response to receiving the ultrasound pulses, and generate localization data based on the return signals detected;
utilizing a display communicating a location of the marker seed based on the localization data to locate the marker seed and perform a medical procedure on the ROI in the subject.

4. The method of claim 3 wherein the medical procedure includes a resection of tissue from the ROI.

5. A kit for localizing a marker seed at least partially composed of a piezoelectric material, comprising:
at least one marker seed including a piezoelectric material;
a detector probe comprising:
a housing extending along a central axis from a distal end to a proximal end;
an ultrasound transducer arranged at the distal end of the housing and configured to:
generate ultrasound pulses into a subject having received the marker seed in a region of interest (ROI);
detect return signals generated by the marker seed in response to receiving the ultrasound pulses; and
generate localization data based on the return signals detected;
a processor in communication with the ultrasound transducer to receive the localization data therefrom and configured to process the localization data to compute a location of the marker seed; and
an output communicating the location of the marker to a user based on the computed location of the marker seed.

6. The kit of claim 5 wherein the output includes a display configured to display a map of the ROI showing the computed location of the marker seed.
